(19) United States
(12) Patent Application Publication
(10) Pub. No.: US 2010/0196302 A1
(43) Pub. Date: Aug. 5, 2010

(54) Keratin Hydrolysates, Process for Their Production and Cosmetic Composition Containing the Same

(76) Inventors: Alane Beatriz Vermelho, Vargem Pequena (BR); Ana Lúcia Vazquez Villa, Recreio dos Bandeirantes (BR); Ana Maria Mazotto de Almeida, Rio de Janeiro (BR); Edilma Paraguai de Souza Dias, Rio de Janeiro (BR); Elizabete Pereira dos Santos, Rio de Janeiro (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/666,409

(22) PCT Filed: Jun. 30, 2008
(86) PCT No.: PCT/BR2008/000180

§ 371 Date: Dec. 23, 2009

(30) Foreign Application Priority Data
Jun. 28, 2007 (BR) PI 0705059-3

(51) Int. Cl. A61K 8/65; C12P 21/06; A61Q 19/00; A61Q 5/12

(52) U.S. Cl. 424/70.1; 435/71.2; 530/357; 514/2

(57) ABSTRACT

This present invention provides a process for keratin hydrolysis by means of microbiological and/or enzymatic processes. In particular, the keratin is derived from feathers of animals, such as chicken and are submitted to hydrolysis by a strain of Bacillus sp. The hydrolysates have molecular weight lower than 500 Da, which makes them ideal for cosmetic applications, particularly for applications in compositions for treatment of hair fiber re-building.

### FIELD OF THE INVENTION

The present invention relates to a process for the hydrolysis of keratin through microbiological and/or enzymatic processes. In particular the keratin is derived from feathers of animals, such as chicken, and is submitted to hydrolysis by a Bacillus sp. strain. The hydrolysates have molecular weight less than 500 Da, which makes them ideal for cosmetic applications, particularly for applications in compositions for re-building treatment of the hair's capillary fiber.

### BACKGROUND OF THE INVENTION

It is known that the keratin has great value in the cosmetics industry because its action on the capillary fiber. The keratin hydrolysates can be prepared by an acid or alkaline hydrolysis, or by enzymatic digestion. The function of chemical or enzymatic hydrolysis is to divide the peptide chains into smaller peptides with lower molecular weights. The keratin source usually used may be from several origins: may be derived from human hair fibers, wool, animal hair, feathers and horns.

The state of the art has several documents relating to keratin hydrolysates, and its use in cosmetics. U.S. Pat. No. 7,220,405 describes a cosmetic formulation which has peptides base, like conditioners and hair dye creme lotion, body moisturizers, skin tone cream and nail enamels. Peptides have been described as being able to connect with high affinity to hair, skin and nails. Thus, this work aimed the development of personal care products containing peptides, from collagen, elastin, soybean, casein, silk among others, coupled directly or via a spacer to the product active agent. The present invention also uses hydrolysates of various proteins, showing the efficiency of these peptides in cosmetics, but does not use keratin of hydrolyzed feather nor the enzymatic method for the proteins cited hydrolysis.

U.S. Pat. No. 4,186,188 describes the use of trypsin to hydrolyse proteins in general, generating polypeptides from 200 to 2000 Da with positive charge that can be used in cosmetic formulations to hair, nails and skin.

Trypsin is a peptidase that has no action on the keratin, thus, it can not be used to hydrolyse keratin. In the present invention keratin hydrolysis is made by keratinases and peptidases with activity on keratin, produced by B. subtilis AMR during fermentation of chicken feathers. So, the enzymatic-microbiological method of the invention presents a greater specificity, because the keratinases acts directly on the keratin polymer.

Documents US 2006/134092 and U.S. Pat. No. 5,395,613 describe peptidases (enzymatic characterization work) produced by microorganisms of the genus Bacillus and Micrococcus sedentarius, able to degrade proteins highly resistant to denaturation and degradation, including keratin, prion (first job) and collagen (second job).

The above patents were aimed at describing a Bacillus or Micrococcus sedentarius peptidase to the degradation of proteins that are difficult to degrade, like keratin. Our work uses another organism, Bacillus subtilis strain AMR keratinolitic, to the degradation of keratin. The goal of our work was not recovering the enzyme, but to use the hydrolysis products to the addition in cosmetic formulations.

U.S. Pat. No. 6,858,215 presents a method for the treatment of hyperkeratinized tissues in mammals using proteolytic enzymes originally developed for the hydrolysis of proteins associated with food and currently is commonly used to soften meat and improve the food taste. The composition of the product developed in this patent has softening enzymes, which soft and exfoliates skin hyperkeratinized formations, as callosities, granules, drying, scaly skin and keratosis without damaging the surrounding tissues by selective lysis of hyperkeratinized tissues. The enzymes used (1 to 15% in the formulation) were the subtilisin Carlsberg and a fungal peptidase of Aspergillus oryzae. The present invention also describes a cosmetic and pharmaceutical product acting on a keratinized tissue. However, in said patent, the enzymatic process aims to hydrolyze the keratin “in situ”, softening hyperkeratinized tissues, as calluses. Our invention uses the enzymatic hydrolysis product of feather keratin through the action of peptidases and keratinases of B. subtilis AMR in cosmetic formulation for hair. The above patent uses enzymes directly on the skin.

U.S. Pat. No. 4,591,497 presents an odor remover and deodorant that contains hydrolyzed material as the effective component consisting of keratin (from 0.1 to 10% with weight of 200-5000 Da) of aminal hair, feathers, nails, hooves, horns and scales. The keratin hydrolysis was achieved by known methods, using acids, alkalis or enzymes. The compound acts effectively on mercaptans and hydrogen sulfide removing its odor. The use of keratin hydrolysates on the above patent has a different purpose of that presented in the present invention. The achievement of this hydrolysate also employs a different methodology. While the above work used acid, alkaline or enzymatic hydrolysis, we used the enzymatic-microbiological hydrolysis.

Documents U.S. Pat. No. 5,262,307, CA 1108542 and U.S. Pat. No. 4,390,525 describe the pretreatment of material containing keratin (feathers, hair, etc) with a reducing agent, or acid treatment, followed by enzymatic digestion of denaturated keratin obtained in the first process phase. In the first patent, the pretreatment is done with sulfite in aqueous solution (to 60-100 degrees) for the denaturation of keratin. In the second, before the enzymatic hydrolysis in the presence of urea, the keratin is submitted to acid conditions at temperature of 80° C. The third patent describes the development of a keratin hydrolysate containing at least two mercaptan groups per molecule and molecular weight between 2,000 to 20,000 Da, suited for cosmetic applications for hair, especially fixers. The hydrolysate is prepared by the reduction of keratin in aqueous solution containing mercaptan and sulfite under alkaline conditions, followed by enzymatic hydrolysis. The oligopeptides reached at the end of the described processes can be used in cosmetics. In the cited document, they made the enzymatic hydrolysis of previously reduced keratin under alkaline conditions or in the presence of sulfite or partially hydrolyzed in acid. In the present invention, hydrolyzed keratin is obtained from the microbial and enzymatic degradation of entire feathers without any prior chemical treatment.

Some patents that deal with the keratin hydrolysis by physical methods are described below. U.S. Pat. No. 5,772,968 describes a system, including equipment and methods, to hydrolyse keratin based materials as carcasses not used, hair, feathers, among others, to obtain protein products useful and marketable. This “hydrolysis system” makes use of an apparatus that promotes the breakdown of keratin by heat, expansion, stirring, mixing and drying. The high temperature added to the mixing process of feathers (keratinolytic material selected for the job), in a conductor tube with screw, leads the hydrolysis of the substrate. The goal of keratin hydrolysis is to increase its digestibility and that it can be used as animal feed supplement. U.S. Pat. No. 4,172,073 describes the keratin hydrolysis obtained of animals structures, using high-pressure saturated steam, finally getting a meal soluble in water and excellent for use in animal feed, since it is digestible by pepsin. These patents describe physical processes (temperature, mechanical action, high pressure) to break the keratin molecule, so that it may have increased their digestibility, in other words, for food use. The present invention provides the microbial and enzymatic hydrolysis of feather keratin, in a process that does not require expenditures for heating, with the aim of implementing the small peptides fragments generated for use in cosmetic hair.

Some patents that deal with the keratin hydrolysis by chemical methods are described below. Document US 2007/0065506 describes the use of keratin and its derivatives (50-60.000 Da) as oral supplement administered to the reduction of oxidative stress and their benefits for promoting the skin health and anti-inflammatory response. The keratin derivatives produced were S-sulfonate keratin intermediate filaments, high sulphur S-sulfonate keratin and peptides of hydrolyzed S-sulfonate keratin. The keratin derivatives were obtained from human hair, wool, animal fiber, hooves and horns, by partial oxidation. The keratin in the patent described above was obtained from mammals, while our keratin source was obtained from chicken feathers. The treatment of keratin in the above patent was chemical, while those of the present invention were enzymatic-microbiological methods and the final product is also completely different.

Document US 2007/128134 presents a keratin derivative soluble in water and its applications. The keratin derivative was obtained by alkaline treatment of feathers followed by exposure to radiation at wavelength of high-energy, leading to obtain peptides with molecular weight between 5,000 to 50,000 Da.

Patent application US 2004/0210039 reports a process to solubilize keratin from materials made of keratin as of chicken feathers. The keratin was solubilized using sulfite under alkaline conditions. In said process the cysteine residues are partially modified by the alkylation and the keratin is partially hydrolyzed. This partially hydrolyzed keratin, with molecular weight between 1,000 and 10,000 Da, can be used for the films production.

U.S. Pat. No. 5,679,329 describes the development of a cosmetic composition containing milk protein (0.02%-15%) and/or hydrolysed milk protein and hydrolyzed keratin (0.1-10%) with molecular weight between 100 to 200,000 Da. The hydrolyzed keratin can be obtained by the hydrolysis of hair, wool, leather, silks, feathers, scales, horns and hooves. In the present invention, the keratin come from moderate acid hydrolysis (fragments of approximately 100,000 Da found in the KERASOL sold by CRODA) and controlled (fragments of approximately 150 Da obtained from the product CROQUAT WKP also sold by CRODA) of bovine hoof. The final product is mainly mousse used to fix hairstyles.

U.S. Pat. No. 5,154,916 describes the addition of keratin hydrolysates with molecular weight of 50,000 in eyelashes mask using a wax as base, improving the properties of coverage, stability and length of the eyelashes. The keratin to hydrolysis may be obtained from hair, wool, hooves, horns, hair, silks and feathers. It was used, mainly, keratin of hydrolyzed skin by moderate alkaline hydrolysis in concentration between 0.05 to 5%.

U.S. Pat. No. 4,839,168 describes the compositions of a hair cosmetic comprising a plant extract (preferably of birch, rosemary and hamamelis) obtained by polar solvent extraction with a polypeptide compound (weighing between 100 to 100,000 Da) including keratin, keratin derivative, silk and hydrolysate of silk. The keratin used is from human hair, wool and feathers and was extracted by oxidation and reduction methods, and hydrolyzed by acid hydrolysis. The product aims to improve a suitable degree of set retentivity and good feeling to the touch.

U.S. Pat. No. 4,818,520 describes the use of keratin hydrolysate, obtained from material as keratinized bird feathers, which may be useful as an anti-skin blemisher, moisturizer, skin mask, shampoo enhancer, shaving lotion and nail hardener and conditioner. The hydrolysate is obtained by heating feather flour in alkaline solution, under reflux, followed by cooling, filtration and acidification with chloridric acid and new filtration, heating and evaporation. Finally, there is a new stage with alkaline treatment followed by neutralization and, thereby, a liquid hydrolyzed keratin is obtained.

U.S. Pat. Nos. 4,465,664 and 4,460,566 describe the composition of hair products containing at least one sort of material derived from keratin, such as animal hair, human hair, wool, nail, feathers, hooves, horns and more. Keratin derivatives were obtained by oxidation (which converts the disulfide bridges to sulfonic acid) or reduction (which produces derivatives with thiol groups) of keratin and used in concentration of 0.01-10% and 0.1-10% respectively, and size between 30,000 to 100,000 Da. The final product of the first patent has moisturizing effect on the hair.

U.S. Pat. No. 3,970,614 reports the solubilization of keratin materials as feathers from chickens, animal hair, hair and hooves, through treatment with N,N-dimethylformamide at high temperature to produce a protein hydrolysate suitable for food use or food supplement for animals and humans and as a food material in food products.

The main difference between the patents described above and the approach of the present invention is the process used for the hydrolysis of keratin; while the patents described above use chemical hydrolysis (acid and/or alkaline, or using N,N-dimethylformamide), or reactions of oxidation and reduction, the present invention uses an enzymatic-microbiological hydrolysis. In addition, the keratin source also differ in some cases. While some works use keratin from bovine hooves and skin, the present invention uses keratin from chicken feathers. The product of the invention uses only hydrolyzed keratin with molecular weight less than 500 Da, which facilitates the absorption and penetration of small peptides fragments in the hair's capillary fiber. The end product also has some purpose other than the above patents.

The article by Roddick (Roddick-Lanzilotta, A & R. Kelly 2006. Protecting the Natural Hair with Keratin Biopolymers. Cosmetics & Toiletries, 121 (5)) describes two strategies for production of intact keratin biopolymers and keratin peptides from wool sheep's for application in hair cosmetics. This polymer connects, preferentially, to sites of the hair fiber protecting it from damage, and contains antioxidant properties. The keratin production involves purification steps to obtain intact keratin (55 kDa), which has properties to form films that can be applied to cosmetics. The peptides of low molecular weight, able to enter the cortex of the hair fiber, were obtained from Croda Chemicals Europe, or obtained by chemical hydrolysis. The obtention of intact keratin cited in the above work, was not the target of the present invention, despite intact feather keratin (approximately 10 kDa) has been found in the supernatant culture of the used microorganism (data not shown). The article cites steps to purify that burden the protein production. The hydrolysate used by them, was obtained by chemical hydrolysis, while that of the present invention was obtained by microbiological hydrolysis.

In the literature, there is a large amount of work reporting the degradation of feathers by keratinolitic microorganisms through submerged fermentation of feathers aiming to use the obtained hydrolyzed keratin as a food protein supplement in the animal diet. However, the present invention aimed at implementing these hydrolysates from microbiological digestion in cosmetic formulations for capillary use, a new purpose for these products from the feathers fermentation. Below are listed the patents, scientific articles and dissertation or thesis using the microbiological hydrolysis of feathers to obtention of keratin hydrolysates for use in ration. None of the documents cited below uses the microbe Bacillus subtilis strain AMR.

The main documents are U.S. Pat. No. 6,214,576, U.S. Pat. No. 5,887,000, U.S. Pat. No. 5,186,961, U.S. Pat. No. 5,063,161 and U.S. Pat. No. 4,908,220. Also, yet other scientific literature is cited as reference:
Maciel, J. L. Produção de hidrolisados protéicos de penas de frango utilizando bactérias queratinoliticas. 2006. Master Dissertation. UFRGS.
Grazziotin A., Pimentel, F. A., Sangali, S., Jong, E. V. and Brandelli, A. 2007. Production of feather protein hydrolysate by keratinolytic bacterium Vibrio sp. kr2. Bioresour. Technol, 98(16): 3172-5.
Geun-Tae Parka, Hong-Joo Sonb, 2007. Keratinolytic activity of Bacillus megaterium F7-1, a feather-degrading mesophilic bacterium. Microbiological Research (in press).
Mio Kojima, M., Kanai. M., Tominaga, M., Kitazume, S., Inoue, A. and Horikoshi, K. 2006 Isolation and characterization of a feather-degrading enzyme from Bacillus pseudofirmus FA 30-01. Extremophiles 10: 229-235.
Brandelli, A. & Riffel, A. 2005. Production of an excellular keratinase from Chryseobacterium sp. growing on raw feathers. Electronic Journal of Biotechnology, ISSN: 0717-3458.
El-Refai, H. A., AbdelNaby, M. A., Gaballa, A., El-Araby, M. H. & Abdel Fattah, A. F. 2005. Improvement of the newly isolated Bacillus pumilus FH9 keratinolytic activity. Process Biochemistry, 40: 2325-2332.
Grazziotin, A., Pimentel F. A., de Jong, E. V. and Brandelli A. 2005. Nutritional improvement of feather protein by treatment with microbial keratinase. Animal Feed Science and Technology, article in press.
Suntornsuk, W. & Suntornsuk, L. 2003. Feather degradation by Bacillus sp. FK46 in submerged cultivation. Bioresource Technologyy, 86: 239-243.
Nam, G., Lee, D., Lee H., Lee, N., Kim, B., Choe, E., Hwang, J., Suhartono, M. T., and Pyun, Y. 2002. Native-feather degradation by Fervidobacterium islandicum AW-1, a newly isolated keratinase-producing thermophilic anaerobe. Arch Microbiol 178:538-547.
Longshaw, C. M., Wrrigght, J. D., Farrel, A. M. &Holland, K. T. 2002. Kyptococus sedentarius, the organism associated with pitted keratolysis, produces two keratin-degrating enzymes. Journal of Applied Microbiology 93:810-816.
Muhsin, T. M., & Hadi, R. B. 2002. Degradation of keratin substrates by fungi isolated from sewage sludge. Mycopathologia, 154: 185-189.
Yamamura, S., Yasutaka, M., Quamrul, H., Yokoyama, K. & Tamiya, E. 2002. Keratin degradation: a cooperative action of two enzymes from Stenetrophomonas sp. Biochemical and Biophysical Research Communications, 294: 1138-1143.
Kim, J. M., Lim, W. J., Suh, H. J. 2001. Feather-degrading Bacillus species from poutry waste. Process Biochemistry, 37: 287-291.
el-Naghy, M. A., el-Ktatny, M. S., Fadi-Allah, E. M. & Nacer, W. W. 1998. Degradation of chicken feathers by Chrysosporium georgiae. Mycopathologia, 14(2): 77-84.
Dalev, P., Ivanov, I. & Liubomirova. 1997. Enzymic modification of feather keratin hydrolysates with lysine aimed at increasing the biological value. J. Sci Food Agric, 73: 242-244.
Singh, C. J. 1997. Characterization of an extracellular keratinase of Trichophyton simii and its pole in keratin degradation. Mycopathologia, 137: 13-16.
Santos, R. M. D. B., Firmino, A. A. P. & Félix, C. R. 1996. Keratinolytic activity of Aspergillus fumigatus fresenius. Curr Microbiol, 33(6): 340-370.
Lin, X., Lee, C., Casale, E. S. & Shih, J. H.1992, Purification and characterization of a feather-degrading Bacillus licheniformis strain. Applied and Environmental Microbiology, 58: 3271-32575.

### SUMMARY OF THE INVENTION

It is one of the objects of the present invention to provide a process for hydrolysing keratin, comprising the steps of:
a) mixing:
   a.1) from 10⁶ to 5×10⁷ cells/mL of at least one specie of B. subtilis bacteria;
   a.2) from 0.1% to 5% w/w of a material containing keratin fibers;
   a.3) from 0.005% to 10% w/w of cultivation medium;
where the pH of the medium is within the range of 7.5 to 8.5;
b) keep the mixture a) in a temperature ranging from 20° C. to 35° C. under agitation;
c) separate the supernatant obtained in b);
d) separate the hydrolyzed peptides of supernatant from c) stage;

In preferred embodiments of the invention, the B. subtilis is the B. subtilis strain AMR.

In preferred embodiments of the invention, the material containing keratin fibers, is chicken feathers.

In preferred embodiments of the invention, the pH is 8.0.

In preferred embodiments of the invention, the cultivation medium comprises yeast extract, peptone, KCl, sucrose or mixture of them.

In preferred embodiments of the invention, the mixture is kept at 28° C. under stirring of 300 rpm.

In preferred embodiments of the invention, the separation of supernatant is made by centrifugation, in particular by centrifugation of 4,000 rpm for 20 min.

In preferred embodiments of the invention, the separation of peptides hydrolysates is made by ultrafiltration.

It is another object of the present invention an optional process of preparation of material containing keratin fibers, including at least one stage of washing and/or delipidation.

In preferred embodiments of the invention, the chicken feathers are washed with detergent and water, dried, delipidated with a mixture of chloroform:methanol and dried.

It is an additional object of this invention, a hydrolyzed keratin with molecular weight in the range from 500 to 1000 Da. In particular, the keratin hydrolysate is obtained by the process of this invention.

It is another object of the present invention, a cosmetic composition comprising a keratin hydrolysate in a concentration ranging from 0.0001% w/w to 20% w/w.

In preferred embodiments of the invention, the cosmetic composition is applied on keratinous tissues such as hair, skin and/or nails.

In preferred embodiments of the invention, the composition is a cosmetic shampoo.

In preferred embodiments of the invention, the composition is a cosmetic cream conditioner.

These and other objects of the invention will be appreciated and better understood by the skilled persons in conjunction with the detailed description below.

### BRIEF DESCRIPTION OF THE FIGURES

FIG. 1 presents the MALDI-TOF of supernatant from B. subtilis AMR culture in the medium after 120 h of cultivation, showing the peptides derived from feather keratin.

FIG. 2 illustrate the MALDI-TOF of CRODA keratin hydrolysates obtained from the pig hooves.

FIG. 3 shows the zymography with gelatin and keratin of supernatant of Bacillus subtilis on feathers demonstrating the presence of proteolytic and keratinolitic enzymes.

FIG. 4 presents the HPTLC of keratin hydrolysates of feather by microbiological degradation. 1-glycine control; 2-peptides of 1000 Da collected by the membrane; 3-peptides of 500 Da collected by membrane; 4-commercial hydrolysate Queratan.

FIG. 5 shows the SDS-PAGE of captured and filtered material by the membrane of 1000 Da by the ultrafiltration process in AMICON. Staining: silver nitrate.

FIG. 6 shows the zymography of captured and filtered material by the membrane of 1000 Da by the ultrafiltration process in AMICON. Staining: coomassie blue.

FIG. 7 presents the employed methodology fluxogram, exemplifying the treatment to which the locks of hair were submitted.

FIG. 8 shows the evaluation of hydration level of the locks of dried hair without heat (room temperature). Locks of hair group: 1-virgin hair; 2-dyed hair; 3-light dyed hair; 4-dyed straightened hair, and 5-completely discolored hair.

FIG. 9 shows the evaluation of hydration level of the locks of dried hair with heat and hair straightener. Locks of hair group: 1-virgin hair; 2-dyed hair; 3-light dyed hair; 4-dyed straightened hair, and 5-completely discolored hair.

FIG. 10 presents the graphic representation of sensory results analysis for the locks of dried hair group without heat.

FIG. 11 presents the graphic representation of sensory results analysis for the locks of dried hair group with heat.

### DETAILED DESCRIPTION OF THE INVENTION

The details and preferred examples reported below are intended to facilitate the reproduction of the invention and should therefore be understood as merely illustrative, without restricting the scope of the invention. Any other way of achieving similar and/or equivalent should be interpreted as within the scope of the invention.

### Material Containing Keratin Fibers

A material containing appropriate keratin fibers in accordance with this invention can be chosen from group comprising natural sources of keratin fibers such as hair, feathers, hooves, nails, horns and similars. Feathers are the preferential material to start, especially chicken, ducks, geese or other birds feathers, such as subproducts of the poultry industry.

The keratin fibers are preferably submitted to a pretreatment such as cleaning, washing, delipidation, cutting, grinding, drying or combinations thereof. This pretreatment aims to facilitate the handling of keratin fibers, which can improve the efficiency of the process and can also influence the quality of the final product.

B. subtilis

The useful bacterium in this invention is the species Bacillus subtilis. In particular, the kind useful in this invention can be chosen among different variants. The best is the variant B. subtilis strain AMR.

### Cosmetic Composition

A technician in the subject can choose the appropriate manner, and also the method of preparation, based on knowledge of the area, taking into account the nature of the ingredients utilized, in particular its solubility, and also the intended use for the composition.

The compositions according to this invention may be in aqueous solution form, water-alcohol mixtures, oily, oil-in-water emulsions, water-in-oil or multiple emulsions, aqueous or oily gels, anidro products, or oily phase in an aqueous phase dispersion comprising nanoparticles, as nanospheres and nanocapsules, or ionic lipid vesicles (liposomes) and/or non-ionic.

When the composition according to the invention is an emulsion, the oily phase proportion can vary, for example, 5% to 80% by weight, preferably from 5% to 50% by total weight of the composition. The oils, emulsifiers and co-emulsifiers used in the composition are chosen among those used conventionally in the corresponding technical field. The emulsifier and co-emulsifier may be present in the composition in a proportion ranging from 0.3% to 30% by weight, preferably from 0.5% to 20% by total weight of the composition.

For hair applications, the composition may preferably be in cream form, lotions, gels, mousses or emulsions, or in aerosols form, including a pressurized propellant. It may be in the form of a lotion to hair care, a shampoo or conditioner, a liquid and/or solid soap, a product to shape the hair (modeling gel, mousse, laque), a colour shampoo, a composition for hair straightening, a sparkling cream. It may also be in hair dye form, to be applied by brush or comb.

For nail applications, the composition can preferably be, a product as a color enamel, a nail base coat, or a product to be applied under or over another product, an enamel remover, or a product to protect, strengthen and/or repair the nails.

For skin applications, preferably, the composition may be more or less viscous, and may have the appearance of, for example, a white or color cream, a skin salve, a lotion or a gel.

The compositions can be applied by any appropriate means, such as brushes, sprays, or with fingers, for example.

The compositions are also associated with procedures for care, treatment, strengthening and/or repair of keratin substrates, where the described composition in this invention is applied to the skin, hair (including lashes) and/or nails, optionally followed by rinsage.

For the purposes of this invention, the expression “keratin substrates care” means a composition directed to improve the appearance and/or the surface of keratin substrates. The care of such substrates can be to make hair smoother and less brittle.

For the purposes of this invention, the expression “strengthening and/or repair keratin substrates” means a composition directed to retain and/or restore the physical and/or mechanical properties of such substrates, which can manifest as such: keratin substrates rigidity, which gives greater consistency and a good feeling to the touch, resulting in an increase volume of keratin fibers and also ease modeling and maintenance of hairstyle; better elasticity and/or resistance to mechanical forces applied, such as during the comb.

The hydrolyzed keratin obtained do not cause interference in the color of the product where it will be applied (shampoo and cream rinse) because it has a clear color, different from many hydrolysates available that have strong tone and may influence the final color of the product. The hydrolysate of this invention can be present in cosmetic formulation at a rate of 0.0001% w/w to 20% w/w.

### Major Advantages

The main advantages of hydrolysate presented in this invention in relation to the state of the art are:
Obtainment of peptides of lower molecular weight;

The use of peptides of 500 Daltons of molecular weight allows greater penetration of peptides in the hair cuticle. The preparations currently on the market have peptides in the range of 980-1300 Daltons.
Usage of clean technology;

The processes biocatalysed by enzymes are less pollutant. The characteristics of these industrial processes and the biodegradability presented by their effluents meet the requirements of ISO 9000 and ISO 14001, which establish standards for the quality of products and guide the characteristics of production processes, giving emphasis to lower energy consumption, to low environmental impact and to an increased quality of products. In this context it is important to note that the enzymatic processing of raw materials results in products of higher added value.
The use of microorganism as direct producers of biocatalyst (enzyme) in the system of keratin hydrolysates production;

The use of microorganisms in systems of keratin hydrolysates production is advantageous because you can easily obtain large populations of microorganisms and therefore more enzyme, reducing the time of degradation of feathers and, in addition, a control of production in all phases. Other advantages are that microorganisms can be genetically improved, allowing an increase of quantity and/or quality of biocatalyst produced and also the choice of high activity strains.
The growth medium is cheap;

The growth medium is cheap due to abundance of cheap raw material in Brazil (agro-industrial waste from the poultry industry). So is a method that still has the potential to take advantage of a major waste generated by Brazilian industrial activities.
It encourages the recycling of materials and also creates productive agreements among companies.

The cosmetics industry can make an important link with the poultry industry for recovery of this waste, avoiding its incorporation into animal feed or polluting the environment. The system of composting that the industry uses from feathers and poultry litter is impossible to absorb all domestic production.

### Example 1

### Preparation of Chicken Feathers

The feathers used in the medium culture were white chicken feathers washed with detergent in running water, dried at 60° C. and delipidated with chloroform:methanol (1:1 v/v) 1 h under agitation of 300 rpm at room temperature. The delipidation was made in a 4 liters Becker with ⅓ of the volume with feathers and 1 L of chloroform solution: methanol 1:1 (v/v). Then the feathers were removed and dried overnight at 60° C. The entire feathers were added in the culture medium as the main source of carbon and nitrogen.

### Example 2

### Microorganism and Culture Conditions

Bacillus subtilis strain AMR was used for the keratin hydrolysates obtainment. This strain was isolated by our waste agro-industrial laboratory of RICA poultry industry and currently deposited in the collection of culture of Oswaldo Cruz Foundation with the registry number of 1266. This microorganism was chosen because of its intense keratinolytic activity for chicken feathers. The bacillus was grown in yeast extract medium (yeast extract 0.5%, 0.5% peptone, KCl 2.0% and 2.0% sucrose) for 2 days at 28° C. under constantly agitation (300 rpm) to obtain a cell mass and washed with saline (2×3000 rpm/20 min) for removal of components of the medium before being inoculated in the medium containing 1% of feathers. Then the cells were transferred to PBS medium pH 8.0 (NaH₂PO₄ 0.06M and K₂HPO₄ 0.04M) with 1% of chicken feathers (prepared according to the procedures outlined in the previous item) and supplemented with 0.01% of yeast extract. The sample was grown in this medium for 5 days at 28° C. under agitation of 300 rpm. At the end of the bacillus fermentation in the medium containing feathers as the main source of carbon and nitrogen, the supernatant was collected by centrifugation at 4,000 rpm for 20 minutes (Mazotto, A M 2005. Bacillus licheniformis AMR's keratinases isolated from poultry industry. 2005. Monographic Work (BS in Microbiology and Immunology)—Institute of Microbiology Professor Paulo de Goes/UFRJ, R10 de Janeiro, 2005).

### Example 3

### Analysis of the Peptides Present in the Culture Medium by MALDI-TOF

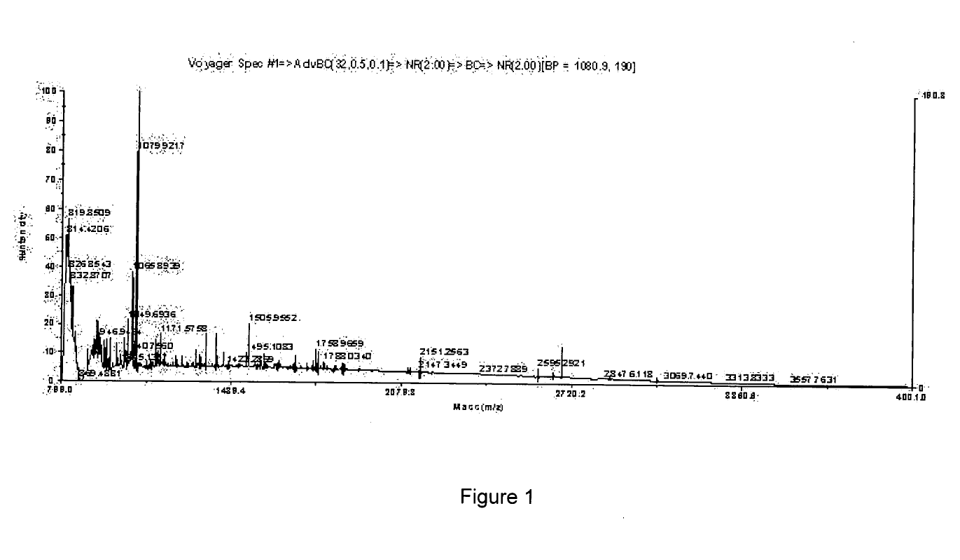

Analyses were conducted in MALDI-TOF (matrix assisted laser desorption/ionisation—Time of flight) for detection of peptides in the culture supernatant (obtained in the previous item) generated by the hydrolysis of feathers by peptidases of B. subtilis AMR. The supernatant containing 4.99 mg/ml of protein determined by Lowry's method (Lowry, O H; Rosembrough, N J; Farr, A L and Randall, R J. 1951. Protein measurement with the Folin phenol reagent. Journal of Biological Chemistry. 193 (1): 267-275) was partially purified in ZipTip C₁₈. The ZipTip C₁₈ was balanced with a solution of acetonitrile (ACN) 100% followed by washing with trifluoroacetic acid (TFA) 0.1%. After this process, the peptides were fixed in the resin ZipTip C₁₈, washed with TFA 0.1% for removal of salts, phosphates and/or DMSO that cause noises during the reading. The elution was made with 0.1% of TFA in ACN 50%. The purified samples were incorporated into the acid matrices of α-cyano-4-hydroxycinnamic (5 μg/ml in TFA 0.1% in ACN 50%) 1:1. The mixture was then applied to the plate for analysis by MALDI-TOF. Analysis in MALDI TOF revealed fragments with molecular weight of 800-1100 Dalton in the crude culture supernatant (FIG. 1).

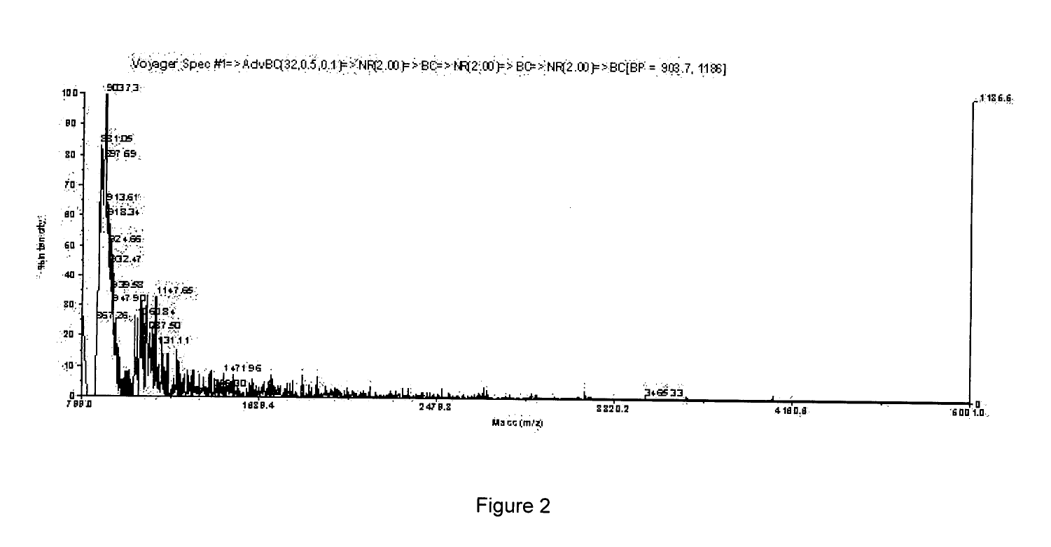

The same procedure of preparation performed for the culture supernatant was also done for the commercial preparation of CRODA diluted 100×. The profile of small peptide fragments observed in the culture supernatant of the bacillus has also been observed in the commercial preparation of CRODA (FIG. 2).

### Example 4

### Separation of Keratin Hydrolysates from Chicken Feathers and Analysis of Amino Acids and Peptides Generated by HPTLC

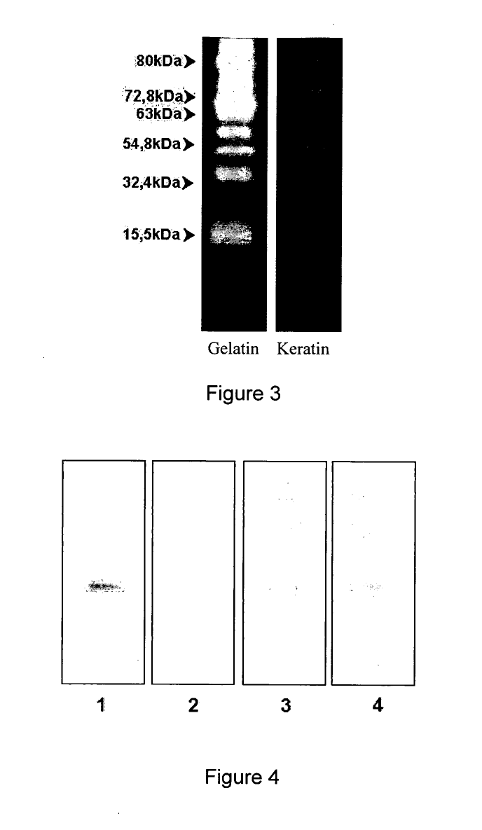

The culture supernatant besides containing the peptides from the hydrolysis of feathers, has also keratinases and peptidases secreted by the Bacillus subtilis AMR. FIG. 3 shows two zymograms with gelatin and keratin in the crude culture supernatant 20× concentrated in the overnight dialysis membrane against polyethylene glycol. The zymographies was prepared according to the methodology described by Heussen & Dowdle (HEUSSEN, C. & Dowdle, E B 1980. Electrophoretic analysis of plasminogen activators in polyacrylamide gels containing sodium dodecyl sulphate and copolymerized substrates. Anal. Biochem, 102:196-202.), however using gelatin (Merck) and keratin extracted from feathers of chicken with DMSO as substrates incorporated to the mesh of polyacrilamide 12.5%.

To obtain a preparation containing only the peptides, the supernatant was submitted to ultrafiltration, using the AMICON system, with the goal of separating peptides up to 500 and 1000 Dalton through membranes of regenerated cellulose Millipore of 500 and 1000 NMWL (Nominal Molecular Weight Limit), respectively. FIG. 4 shows these peptides and amino acids and compares them to a commercial preparation of hydrolyzed keratin from feathers called Queratan (POLYTECHNO) through HPTLC method (high performance thin layer chromatography) and revealed with ninhydrin, showing that the hydrolyzed keratin from feathers by B. subtilis AMR has a pattern of amino acids and peptides similar to the commercial preparation used. It is also observed that the Queratan has, in addition, peptides with higher molecular weight (FIG. 6, line 5). Comparatively, our procedure is better because it has only low molecular weight peptides, which has a greater penetration in the capillary fiber. A solution of glycine was used as a control (1 mg/ml), an amino acid present in high concentrations in feathers, 76.6 g/kg (DALEV, P. 2000. Utilization of biodegradable collagen and keratin-containing wastes through enzymatic treatment. Wolfsburg. Orbiti Association). In HPTLC were applied 10 μL of peptides separated from supernatant with membrane of 500 and 1000 Da.

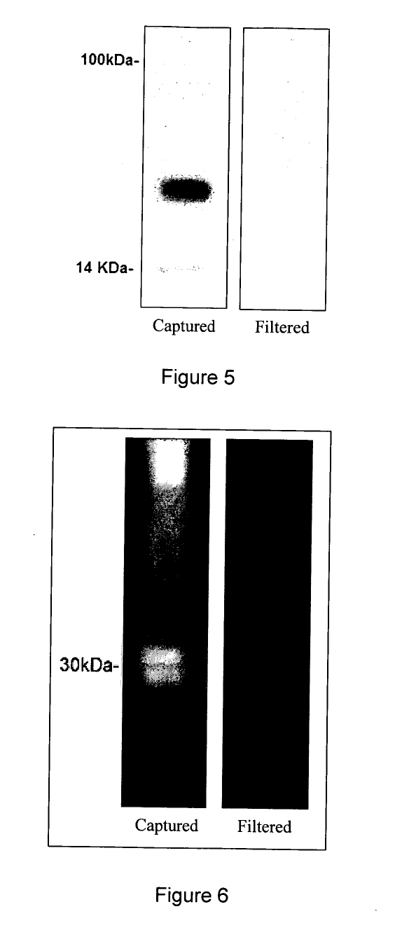

During the ultrafiltration, basically proteins with molecular weight above their exclusion limit were retained in membranes, including the enzymes and solubilized keratin molecules, but not hydrolysed (with molecular weight of approximately 10 kDa). The retained on the membrane and peptides smaller than their limit of exclusion (filtered) were examined by SDS-PAGE (LAEMMLI, VK 1970. Cleavage of structural during the assembly of the head of bacteriophage T4. Nature, 227:680-685.) and stained by the silver nitrate method (FIG. 5) and zymography (HEUSSEN, C. & Dowdle, E B 1980. Electrophoretic analysis of plasminogen activators in polyacrylamide gels containing sodium dodecyl sulphate and copolymerized substrates. Anal. Biochem, 102:196-202.), as shown in FIG. 6. The SDS-PAGE showed only the presence of proteins retained by the membrane (30 μL of sample were applied), an expected result, since peptides and amino acids (60 μL of sample were applied) are outside the limits of detection of the art, the same is true for zymography. This method showed that there was loss of activity of the culture supernatant during the process of ultrafiltration, when compared to the zymography of supernatant before ultrafiltration (FIG. 3 and FIG. 6). The SDS-PAGE and zymography showed the complete absence of proteins in peptidic fraction confirming that there was no contamination of the hydrolysate with proteolytic enzymes and proteins, respectively.

### Example 5

### Cosmetics Application of Hydrolysate Peptides from Chicken Feathers

The peptides of molecular weight smaller than 500 Dalton obtained through ultrafiltration in AMICON (500 NMWL membrane), were incorporated into two cosmetic bases: a gentle shampoo and rinse-conditioner, both containing 10% of the material filtered through the 500 NMWL membrane from AMICON.

Gentle Shampoo with 10% of Microbial Hydrolysate from Chicken Feathers Containing Peptides Smaller than 500 Da

Sodium laureth sulfate . . . 30%

Decyl polyglucose . . . 5%

Lauryl polyglucose . . . 5%

Surfax acid . . . 3%

Coconut fatty acid diethanolamide . . . 4%

Phenochen or Phenova . . . 0.5%

Germal 115 . . . 0.2%

Keratin hydrolysate . . . 10%

Unistab 569 . . . 0.2%

Essence of anise . . . 0.5%

Distilled water q.s.p . . . 100 ml

The Germal requires heating (80° C.) to its dissolution in distilled water. After the dissolution of Germal, the other components were added to the solution. The polyglucose must be heated to incorporate the previous solution. The homogeneity should be done slowly to avoid foaming. Finish adding the microbial hydrolysate from feathers, the Unistab 569 and essence. Complete the final volume, homogenizing.

Rinse-Conditioner with 10% of Microbial Hydrolysate from Chicken Feathers Containing Peptides Smaller than 500 Da

Oily Phase

Cetearyl Alcohol . . . 5%

Phenova . . . 0.5%

Cetrimonium chloride . . . 0.5%

Aqueous Phase

Germal 115 . . . 0.2%

Essence of anise . . . 0.5%

Colour . . . 0.2%

Keratin Hydrolysed . . . 10%

Distilled water qsp . . . 100 mL

All components of oily phase were heated (75° C.) together and homogenized until complete dissolution. In another container, the germal was dissolved under heating to 80° C. After achieving the temperatures, the oily phase was added to water phase, under agitation, until emulsifying. The solution obtained was placed in cold bath, homogenized and increased of other substances of the aqueous phase.

These products were applied in locks of virgin and chemically treated hair, previously washed and defatted for 5 weeks, subject to a treatment with mild shampoo and conditioner, containing the microbial keratin hydrolysate, followed by hair-drying and hair straightener of 180° C. and room temperature, with the aim of assessing the degree of capillary fiber hydration.

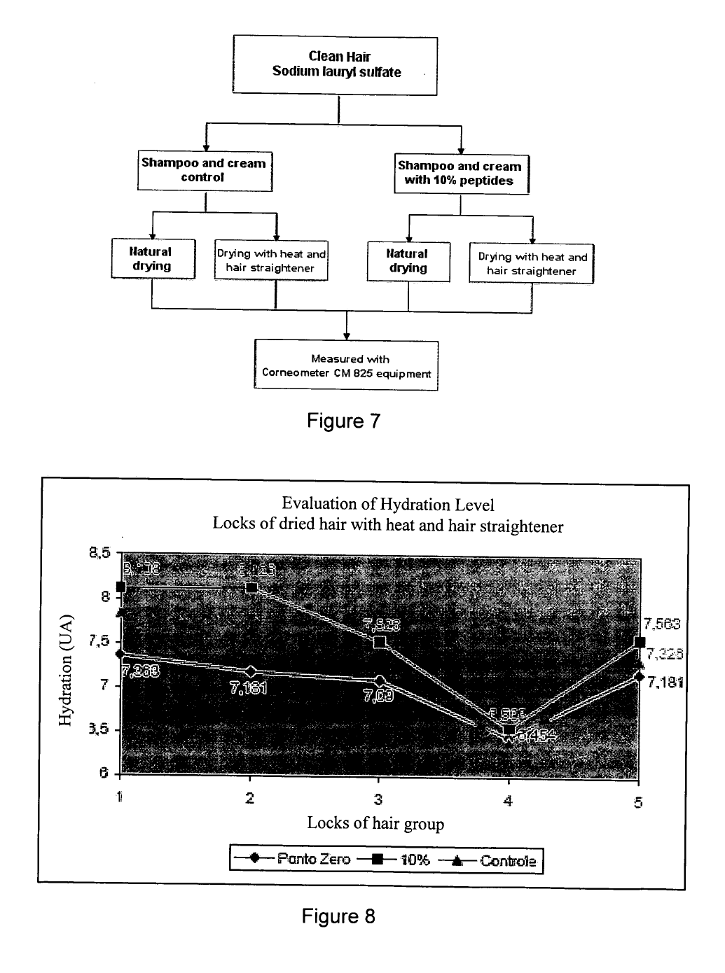

The hair locks were separated on 5 different groups. These are composed of virgin hair, dyed, dyed straightened hair, lights dyed hair (discoloration) and totally discolored. Each group contains four hair locks: two with 10% of keratin hydrolysates and two hair locks controls. Before the test all the hair locks were properly washed with shampoo of sodium lauryl sulfate 2%, and rinsed with distilled water. This procedure was intended to remove any material adsorbed to the string, avoiding interference in the trial. Below is illustrated the flowchart of the methodology used in the process of treatment and measurement of hydration (FIG. 7).

Tests for hydration measurement were performed by Corneometer CM 825 equipment. Control tests of cosmetic base without peptides hydrolysates from chicken feathers were conducted in parallel in the same locks of hair group. Sensory test was applied to evaluate the hair brightness and softening.

### Example 6

### Measurement of Hydration

The process of hydration measurement of hair locks was held every seven days over a period of five weeks. For this, the device used was Corneometer CM 825. The function of this device is to assess the amount of water evaporated by the capillary fiber through field variation. The method is based on the great variability of the dielectric constant of water steam, which is automatically registered by the device. The measured value is given on Arbitrary Unit of Moisturizing (AU), being used for the measurement of hair locks. The temperature and humidity of the environment have always been monitored and they were around 25° C. and 42% humidity, respectively.

For each group, 5 hydration measurements were made and the mean of each assessment was used to show and to discuss the results. The analysis of variance ANOVA was used to statistically measure the final content of hydration in the hair structure with hydrolysate peptides of keratin from chicken feathers, in relation to the formulation without the hydrolysate, in other words, control group.

The degree of hydration among different types of hair locks after treatment can be seen below: (Table 1)

TABLE 1
Content of hydration obtained by each type of hair.

| | Types of treatment | | | | |
|---|---|---|---|---|---|
| Locks of hair group | A | B | C | D | E |
| 1. Virgin | 7.363 | 8.236 | 8.108 | 7.454 | 7.836 |
| 2. Dyed | 7.181 | 8.145 | 8.126 | 7.017 | 7.654 |
| 3. Lights Dyed Hair | 7.09 | 7.781 | 7.526 | 7.017 | 7.29 |
| 4. Dyed Straightened Hair | 6.454 | 7.761 | 6.563 | 7.09 | 6.399 |
| 5. Totally Discolored | 7.181 | 7.672 | 7.563 | 7.036 | 7.326 |

A - zero point lock of hair (only washed with sodium lauryl sulfate) and dryed without heat (naturally); B - lock of hair washed with shampoo and conditioner of hydrolysate peptides of protein 10% and dried with heat and hair straightener; C - lock of hair washed with shampoo and conditioner of hydrolysated peptides of protein 10%, and dried without heat (naturally), D - lock of hair control (washed with control shampoo and conditioner) dried with heat and hair straightener; E - lock of hair control (washed with control shampoo and conditioner) dried without heat (naturally). These averages results were obtained at the end of five weeks.

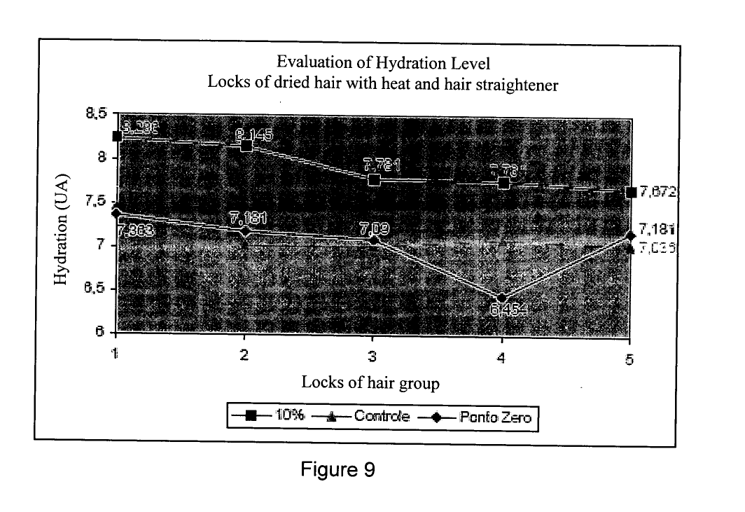

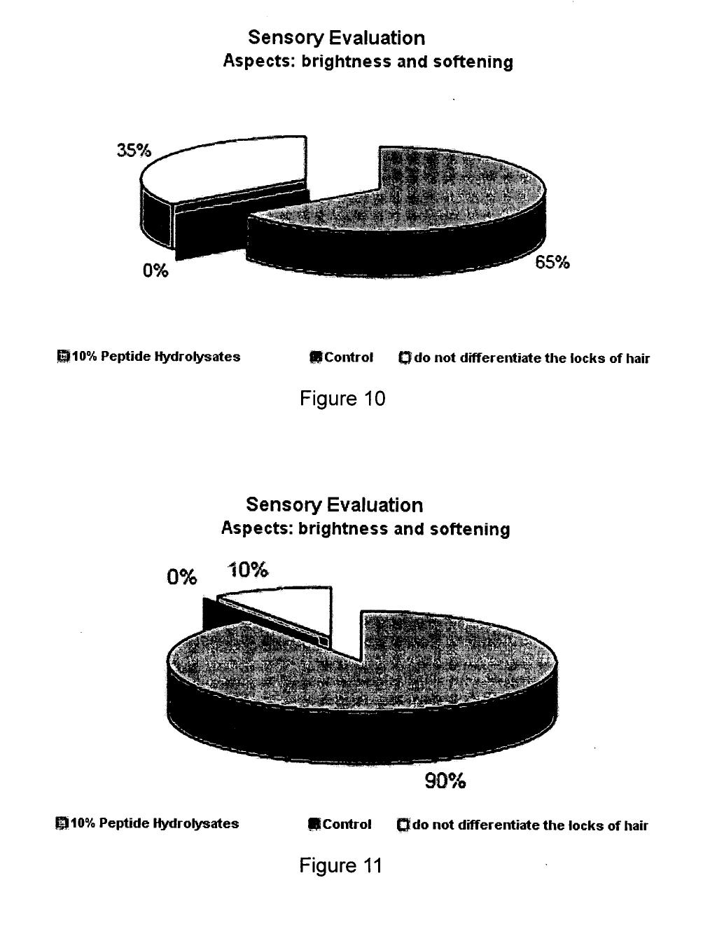

FIGS. 10 and 11 represent the average of the hydration results of the group of locks dried without heat (FIG. 8), with heat and hair straightener (FIG. 9). In both situations comparisons were made between the level of hydration of control locks, zero point and with hydrolysate peptide of protein 10%.

It was observed that the group of locks treated with control preparation did not produced significant hydration when compared with locks treated with hydrolysate peptide of protein from chicken feathers 10%. The average of hydration obtained by the group of locks treated with the control formulation during the two assessments (drying with heat and without heat) was lower than the group treated with the peptide hydrolysate 10%. Including, the locks that were submitted to heat obtained a lower degree of hydration than those dried naturally at room temperature. This finding confirms that the basis used in the formulation did not interfere in hydration. Thus, it becomes clear the importance of the peptidics components (keratin hydrolysate) in capillary fiber structure, providing a greater degree of hydration. This is due to the function that these components have to protect, restore or enhance the hair fibers rendering them more hydrated, because the fragments of amino acids and poli peptides of hydrolysed proteins that are composed of a cluster of amino acid and presents low molecular weight, seem to connect the keratin of hair, restoring the damaged protein structure (PAOLA, MVRV Ethnic hair. Cosmetics & TOILETRIES, Sao Paulo, Brazil, v. 11, p. 36-44, May/June 1999).

For statistical analysis of the results, the method of analysis of variance ANOVA was employed, from which it was possible to observe that the comparisons between the zero point hair locks with the control hair locks with heat and hair straightener and without heat, dried naturally (room temperature) did not show significant results at 5%; the hair locks with the peptides hydrolysate without heat (dried naturally) did not also show a significant result. But the hair locks that used the hydrolyzed peptide of protein 10% had a significant level of hydration at 5%. This consistently confirms the results obtained previously by the method of averages.

Through the results it was observed that the virgin hair—which has its cuticle intact—obtained a greater degree of hydration since it allows the remaining of the product in its structure. In relation to chemically treated hair, it was observed that the level of absorption is greater when the level of aggression in its capillary structure is lower (EXPÓSITO, F. Tricologia. Spain, Madrid, ed. Eldesa, 50-58 p, 1995).

Discoloration or treatment with products for permanent waves decreases the amount of cystine in the capillary fiber. Drastic discoloration also leads to a loss of tyrosine and methionine (Scanavez, C. 2001. Change in hair ultrastructure induced by daily care and its effects on the color properties. PhD thesis under the guidance of Prof. Inês Joekes. Institute of Chemistry—UNICAMP). The use of products with keratin hydrolysates can minimize this loss and improve the characteristics of hair.

The test method used is designed to check the degree of hydration of the skin surface and how the evaluation was made to ascertain the degree of hydration in the hair shaft, because it may have occurred any interference in the results, since the capillary structure show differences in relation to the skin, such as a lesser hydric degree (PEYREFITTE, G.; MARTINI, M.; CHIVOT, M. Biologia da Pele. Estética-Cosmética; Cosmetologia; Biologia Geral, São Paulo: Andrei Organization Ltd., 373-379 p, 1998. BOTELHO, A, J. Avaliação da hidratação capilar em cabelos alisados utilizando preparação cosmética com extrato de algas marinhas. 2006. Monographic work f. 61 (Degree in Pharmacy)—Estácio de Sá. University, R10 de Janeiro, 2006). Further analysis of the capillary structure through electron microscopy can be conducted to confirm the results obtained.

### Example 7

### Sensory Analysis

Test for sensory evaluation of hair locks was also made to evaluate the degree of brightness and softening. The evaluators (total of 20 people) examined the hair locks (zero point) with control and those who were tested, to determine which of them had greater brightness and softening. It was a blind evaluation, because the evaluators did not know which formulation was being used. Before the test began, the evaluators went through a process of washing hands, for withdrawal any grease product that could interfere in the analysis, after that, they responded which group had greater or lesser degree of brightness and softening.

Observing the sensory evaluation conducted to ascertain which of hair locks (with hydrolysate 10% and control) had greater brightness and softening, different results were obtained in accordance with the type of drying performed. For the group of hair locks dried without heat (room temperature) it was observed that the greater percentage of evaluators' preference (65%) was the hair locks that had been treated with peptide hydrolysate of protein 10%; and a lower (35%) was not able to choose which of the groups had greater brightness and softening (FIG. 10). In contrast, for the group of hair locks dried with heat and hair straightener was observed that the greater percentage of evaluators' preference (90%) was the hair locks that had been treated with the peptide hydrolysate 10%; and a lower (10%) was not able to choose which of the groups had greater brightness and softening (FIG. 11). These results were fundamental to assess how the use of hydrolysate peptide of protein interfered in capillary structure making it silkier and brighter.

In the consumer point of view, brightness is directly related to a healthy hair (SCHUELLER, R.; ROMANOWSKI. Q. Assessment of Hair Brightness. Cosmetics & Toiletries, Melrose Park Ill., States U.S., v. 14, p. 52 to 56, September/October 2002).

By using cosmetic products for hair hydration, they are responsible for increasing the amount of water in the string or form a barrier that obstructs the loss of water because the moisture content of hair influence on physical properties as: static load, stiffness, brightness and volume (POZEBON, D. Análise do cabelo: uma revisão dos procedimentos para a determinação de elemento taçõs e aplicaçÕes. Química Nova, Florianopolis, v. 22, n. 6, p. 830-840, 1999. BOTELHO, A, J. Avaliação da hidratação capilar em cabelos alisados utilizando preparação cosmetica com extrato de algas marinhas. 2006. Monographic work f. 61 (Degree in Pharmacy)—Estácio de Sá. University, Rio de Janeiro, 2006).

1. Process of keratin hydrolysis characterized by comprising the steps of:
a) mixing:
a.1) from about 10⁶ to about 5×10⁷ cells/mL of at least one species of bacteria B. subtilis;
a.2) from about 0.1% to about 5% w/w of a material containing keratin fibers;
a.3) from about 0.005% to about 10% w/w of culture medium;
where the pH of the medium is within a range from 7.5 to 8.5;
b) keeping the mixture a) in a temperature within a range from 20° C. to 35° C. under agitation to obtain a supernatant;
c) separating the supernatant obtained in b);
d) separating hydrolysated peptides of supernatant of stage c).

2. The process of claim 1, wherein said bacterium is B. subtilis strain AMR.

3. The process of claim 1, characterized by further using a concentration of 10⁷ cells/mL of bacteria in step a.1).

4. The process of claim 1, wherein the material containing keratin fibers is selected from the group consisting of hair, feathers, hooves, nails, horns and combinations thereof.

5. The process of claim 4, wherein the feathers are chosen from the group consisting of chicken, hen, ducks, geese feathers and combinations thereof.

6. The process of claim 1, wherein the material containing keratin fibers are at a concentration of 1% w/w.

7. The process of claim 1, wherein the culture medium comprises yeast extract, peptone, KCl, sucrose or combinations thereof.

8. The process of claim 1, wherein the growth medium is at a concentration of 5% w/w.

9. The process of claim 1, wherein the process is conducted at pH 8.0.

10. The process of claim 1, wherein the process is conducted at 28° C.

11. The process of claim 1, wherein the process is conducted at an agitation of 300 rpm.

12. Process, according to claim 1, characterized by separation mentioned in c) being held by centrifugation.

13. The process of claim 1, wherein the separation mentioned in d) is conducted by ultrafiltration.

14. The process of claim 1, characterized by comprising an additional step of preparation of the material containing keratin fibers, before using it in the step a).

15. The process of claim 14, characterized by comprising at least one additional step selected from the group consisting of:
A) washing;
B) drying;
C) delipidation, and
D) combinations of a), b) and/or c).

16. The process of claim 15, characterized by the fact that the step of washing is conducted with the aid of a detergent.

17. The process of claim 15, wherein the step of delipidation comprises the use of a chloroform:methanol mixture at a ratio from 0.5:1 up to 3:1.

18. Keratin hydrolysates characterized by having a molecular weight within a range from 500 Da to 1000 Da.

19. Keratin hydrolysates, according to claim 18, characterized by being obtained with the process of claim 1.

20. Cosmetics composition characterized by comprising: a) from 0.0001% w/w to 20% w/w of a keratin hydrolysate having a molecular weight within a range from 500 Da to 1000 Da; and b) an appropriate cosmetic vehicle.

21. The cosmetics composition, of claim 20, characterized by comprising 10% w/w of a keratin hydrolysate.

22. The cosmetics composition, of claim 20, characterized by being formulated for use in hair, skin and/or nails.

23. The cosmetics composition, of claim 22, wherein the composition is in the form of a shampoo and/or conditioner.

24. The cosmetics composition, of claim 20, characterized being formulated for use in the treatment of capillary fiber.
